(19) United States
(12) Patent Application Publication
(10) Pub. No.: US 2013/0280134 A1
(43) Pub. Date: Oct. 24, 2013

(54) STERILIZATION CONTAINER

(71) Applicants: Michael D. Hoffman, Jupiter, FL (US); Franklin Goldberg, Delray, FL (US)

(72) Inventors: Michael D. Hoffman, Jupiter, FL (US); Franklin Goldberg, Delray, FL (US)

(21) Appl. No.: 13/868,653

(22) Filed: Apr. 23, 2013

Related U.S. Application Data
(60) Provisional application No. 61/636,850, filed on Apr. 23, 2012.

(51) Int. Cl. A61L 2/07

(52) U.S. Cl. CPC A61L 2/07
USPC 422/114

(57) ABSTRACT

A sterilization container having a lid and a bottom. The lid is provided with a plurality if bimetal valves that will fully open and fully close at a preset temperature. The lid also includes a pressure equalization valve to relieve pressure from the container that will occur during the heating and cooling process. The bottom and the lid are formed from light weight and highly durable material so provide an easily handled and long lasting apparatus. Each lid includes a plurality of recesses and each bottom includes a plurality of complimentary projections such that multiple containers can be safely stacked upon one another.

### PRIORITY CLAIM

In accordance with 37 C.F.R. §1.76, a claim of priority is included in an Application Data Sheet filed concurrently herewith. Accordingly, the present invention claims priority under 35 U.S.C. §§119(e), 120, 121, and/or 365(c) to U.S. Provisional Patent Application 61/636,850 entitled “STERILIZATION CONTAINER”, filed on Apr. 23, 2012, the contents of which is incorporated herein by reference.

### FIELD OF THE INVENTION

The invention relates to the field of flash and terminal sterilization and, in particular, to a sterilization container used for autoclaving of medical devices.

### BACKGROUND OF THE INVENTION

Prior to medical and surgical procedures, medical devices and surgical instruments must be sterilized to reduce the risk of infection. Various sterilization techniques are utilized in the medical field, such as irradiation of equipment, treatment using antimicrobial solutions, and temperature sterilization techniques.

One of the most commonly employed techniques is sterilization using an autoclave, which involves placement of the medical devices to be sterilized within a chamber which is subsequently heated with steam to a sufficient temperature and time period to kill microbial agents which may be present on the equipment. Within an autoclave, once the chamber is sealed with the instruments needed to be sterilized, all the air is displaced from the chamber by pumping in steam to force the air out, called gravity displacement. Steam is pumped through the chamber at a pressure of at least 15 psi and at a temperature of about 121-140° C. (250-284° F.). The steam pumping continues for a minimum of about 3 minutes and a maximum of about 15-20 minutes, higher temperatures will allow for shorter sterilization times. The exact sterilizing time depends on a variety of factors, including how the autoclave is loaded wherein the free movement of steam allows autoclaving to be quicker and more effective. High Vacuum autoclaves use a vacuum pump to remove air prior to the introduction of steam that creates high pressures within the chamber. This autoclave utilizes several cycles of vacuum and pressure to assist the saturation of steam to travel into the intricate areas of the instruments.

Steam sterilization units, whether they are gravity steam, or alternating vacuum and pressure or the like, all normally have a drying cycle. During the drying cycle, steam is applied to the jacket of the autoclave to create a hot environment and normally some vacuum is applied to the chamber in order to lower the boiling point of the moisture. The drying cycle is utilized to evaporate the moisture in the sterilization container.

Sterilization time depends on the temperature; however, in the medical industry time is a luxury that cannot be depended upon. In many instances the necessity for a quick sterilization demands the use of flash sterilization wherein the medical instruments are exposed briefly to steam a temperature higher than that normally used in autoclaves so that the medical instruments can be returned to use faster. Flash sterilization is accomplished by filling an autoclave with saturated steam at an elevated pressure, e.g. 30 psig, wherein the medical instruments can be returned to use almost immediately.

A problem also exists if the medical instruments are not immediately used or must be transported to a place of use. In order to prevent contamination before use, it is preferred that the medical instruments are kept in a closed container.

Typical sterilization containers are constructed of a lid and base portion, and a vent portion located on the lid, the base or both. The vent portion permits the movement of gases to accommodate the changes in pressure created by increasing or decreasing temperatures of the internal and external environments of the container. In order to prevent contamination by the handling and storage of sterilization containers, filters can be positioned in relation to the vent portion to permit the transport of gases but reduce or prevent the transport of microbes.

U.S. Pat. No. 4,783,321 discloses a sterilization container system for sterilizing surgical instruments. The container has a lid secured to a base by latches. The latches are automatically disengaged when release arms are raised to form handles, the latches automatically engage when the latches' release arms are lowered for sealing the lid to the base. The base is provided with an outer safety wall, which is lower than and which extends around the base's inner wall, in order to help protect the top of the base's inner wall from inadvertent contamination when the lid is removed. Disposable paper filters are sealed over the vents inside the lid and base by cooperating filter paper sealing flanges provided in the filter papers' retainers, lid and base, in order to maintain the sterility of the contents of the sterilization container system after sterilization.

U.S. Pat. No. 5,324,489 discloses a medical instrument sterilization container which includes a port having a removable filter within the port to enable sterilization of instruments placed within container. A removable plug replaces the filter to enable the container to be used to transport instruments without leaking of fluids therefrom.

U.S. Pat. No. 5,968,459 discloses a flash sterilization container formed from a pan that holds a tray, a cover forming the top of the container, and a filter element. The filter covers an opening in the cover so that the inside of the container can communicate with the atmosphere surrounding the sealed container allowing steam to enter and exit the container passing through the filter. Items to be sterilized are placed on the tray and the cover is attached and sealed to the pan. The filter allows steam to enter the interior of the container and sterilize any items contained therein. After sterilization, the container is removed from the autoclave and any remaining steam escapes from the interior of the container through the filter.

U.S. Pat. No. 6,622,862 discloses a container system for flash and terminal sterilization. The sterilization container includes a lid and a tray. The lid has a large aperture and small attachment apertures. A support is formed with an interior central circular disk. An exterior circular ring is also provided. Ribs connect the ring and the disk. The disk has a central aperture. Small attachment apertures are provided for coupling the upper surface of the ring to the lower surface of the lid wall. The spring and plate are adapted to expand when heated to between 135 and 272 degrees Fahrenheit. In this manner a space is created between the plate and the disk allowing steam to enter the chamber whereby medical instruments within the container may be sterilized. The spring and plate are adapted to close when cooled to above about 135 degrees Fahrenheit. In this manner any space between the plate and the disk will be eliminated.

At present there is a need for an improved sterilization container that allows for flash sterilization, provides portability and reduces the likelihood of contamination both during and following sterilization.

### SUMMARY OF THE INVENTION

The present invention is comprised of a bottom tray that is generally rectangular in configuration. The bottom tray has a rectangular horizontal bottom wall and four rectangular vertical upstanding walls. The walls have an upper periphery that that define the opening for the tray. The tray is sized and configured to receive medical instruments and the like to be sterilized. The invention also includes a lid that is generally rectangular in configuration having a rectangular upper wall and four rectangular vertically depending walls. The walls terminate in a lower peripheral edge with an open bottom that is positionable on the upper periphery of the bottom. The lid includes a generally rectangularly shaped recess formed in the upper surface of the rectangular top wall. The rectangularly shaped recess has two large apertures and one smaller aperture positioned between the two larger apertures. A bimetal thermally responsive valve is positioned in each of the large apertures. The number and size of the larger aperture will vary based on the volume of the container. The bimetal thermal responsive valves will fully open and fully close at a preset temperature in a step function manner. The quick and complete closure of the bimetal valves ensures that the apertures will be effectively sealed prior to removal from the autoclave. A pressure equalizing valve (or valves) is positioned in the smaller aperture. The pressure equalizing valve is provided to high pressure relief pressure for the container while in the high vacuum autoclave. The lid further includes a top cap mounted in the rectangular recess that is positioned above the bimetal valves and the pressure equalizing valve. The top cap will serve as a shield or guard for the bimetal valves and the pressure equalizing valve.

The tray and the lid are formed from light weight and highly durable material to provide an easily handled and long lasting apparatus. Each lid includes a plurality of recesses and each tray includes a plurality of complimentary projections such that multiple containers can be safely stacked upon one another. The bottom further includes a pair of handles to facilitate the conveyance of the container from one location to another. A gasket is provided between the lid and the bottom to provide an effective seal for the container when the lid is secured to the tray.

Accordingly, it is an objective of the instant invention to provide a more effectively sealed sterilization container. The container is so sealed that the cooling process creates a significant partial vacuum inside the container sufficient to create difficulty in opening the container. The pressure equalizing valve is equipped with a manual button to relieve this vacuum so the lid can be removed from the bottom.

It is a further objective of the instant invention to provide a flash sterilization container that will ensure container closure prior to removal from the autoclave thereby limiting the potential for contamination.

It is yet another objective of the instant invention to provide a container that is light weight and durable to facilitate ease of use and long term field operation.

It is a still further objective of the invention that to provide terminal sterilization containers that can be easily stacked for storage, transport and use.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of the sterilization container with the lid sealed to the tray.

FIG. 2 is perspective view of the sterilization container with the lid removed from the tray.

FIG. 3 is an exploded perspective view of the sterilization container lid.

FIG. 4 is an end view of the sterilization container lid.

FIG. 5 is an end view of the sterilization container bottom.

### DETAILED DESCRIPTION OF THE INVENTION

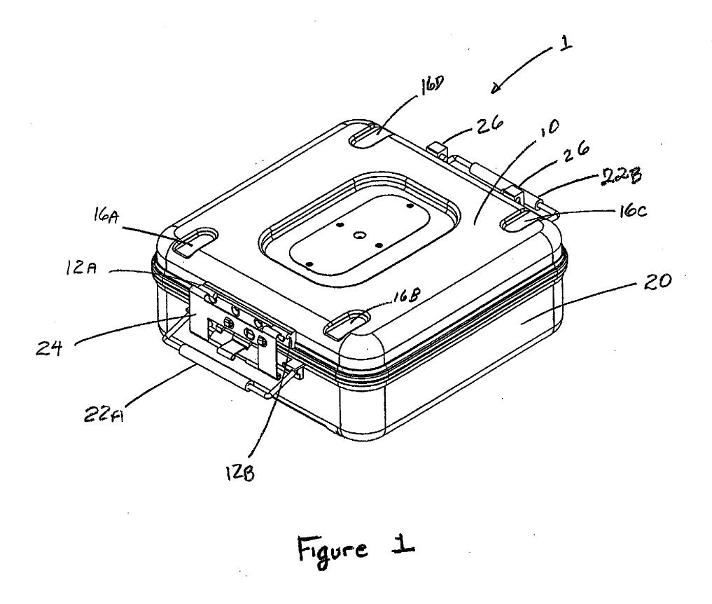

The flash sterilization container embodying the invention includes an upper lid component and a lower bottom component. FIG. 1 is a perspective view of the flash sterilization container 1, in a sealed condition, having an upper lid component 10 and a lower bottom component 20. The bottom 20 includes a pair of handles 22A and 22B located on opposite sides of the tray. A latching mechanism 24 is position on one side of the sterilization container 1 and is pivotally mounted on bottom 20. The latching mechanism 24 includes a pair of flanges that are sized and configured to engage horizontally oriented pins 12A and 12B that are positioned on the lid 10. A hinge arrangement is positioned on the sterilization container 1 on the side opposite the latching mechanism 24. The hinge arrangement includes a pair of “C” shaped arms 26 extending upwards from the tray 20 that can be positioned into operative engagement with horizontally oriented pins 14A and 14B located on lid 10. The pins 12A and 12B are positioned symmetrically with respect to pins 14A and 14B such that the position of the lid with respect to the tray is interchangeable between either of the two possible positions. In this way pins 12A and 12 B can engage latch mechanism 24 or “C” shaped arms 26, and likewise, pins 14A and 14B can engage latch mechanism 24 or “C” shaped arms 26 depending on the orientation of the lid 10 with respect to the tray 20. The container 1 may also have an identical latching mechanism opposite the latching mechanism described above. The lid 10 has four recesses 16A, 16B, 16C and 16D formed on the upper wall. Theses recesses 16A though 16D are sized and configured to cooperate with four protrusions formed on the bottom of the bottom 20 to facilitate stacking of plural containers one upon another. This stacking attribute is a feature that enhances the ability to store, transport and use the sterilization containers.

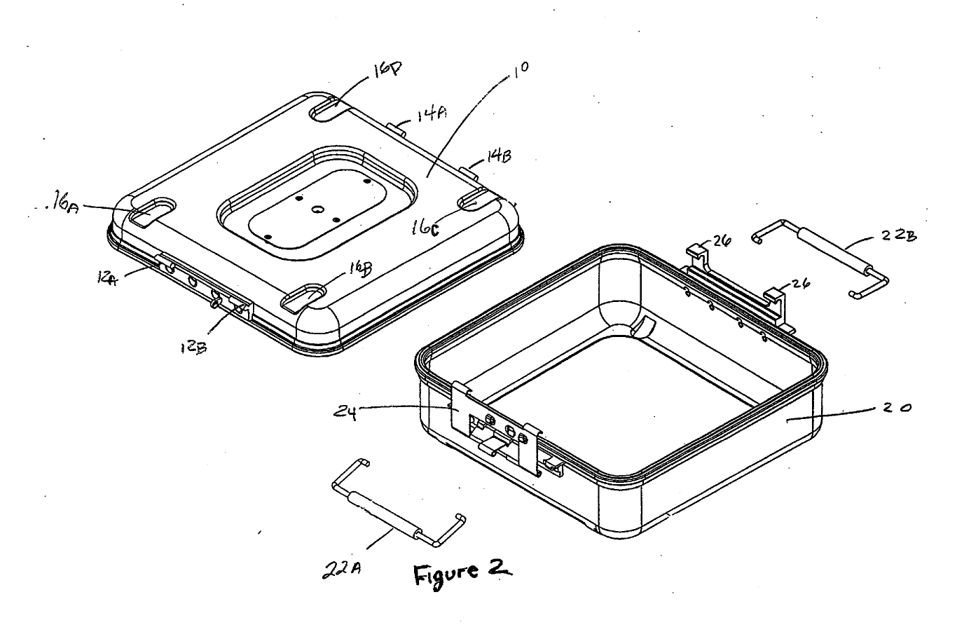

FIG. 2 is a perspective view of the flash sterilization container 1, in an open condition, with the handles 22A and 22B removed for clarity. As shown therein the bottom 20 includes a pair of handle assemblies 22A and 22B as well as latching mechanism 24 and “C” shaped arms 26 of the hinge arrangement. The lid 10 includes horizontally oriented pins 12A and 12B that cooperate with latching mechanism 24 as well as horizontal pins 14A and 14B for the hinge arrangement. Container 1 can also have two identical latching mechanisms on opposite sides.

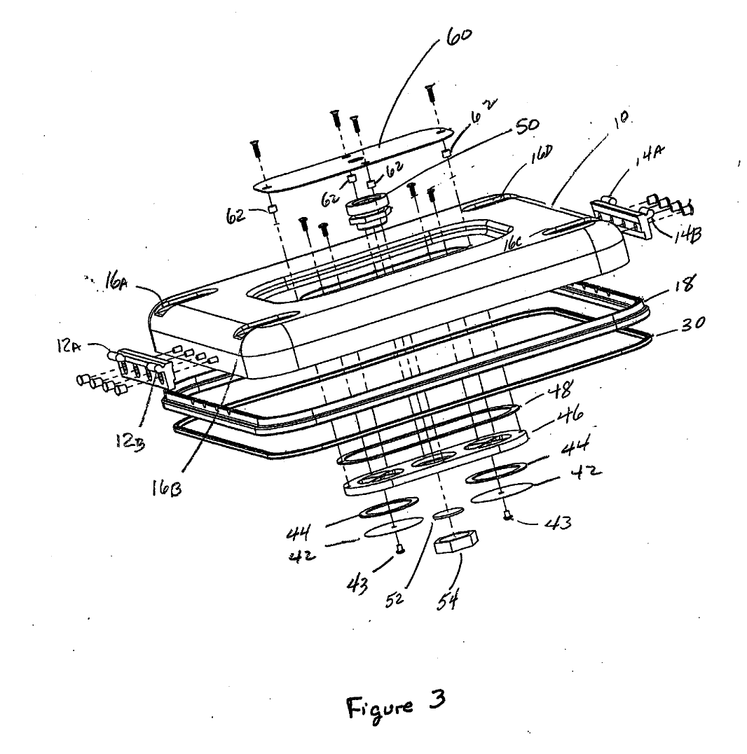

FIG. 3 is an exploded perspective view of the lid 10. As shown therein horizontally oriented pins 12A and 12B are integral with a base member that is secured to lid 10 with a plurality of fasteners. Horizontally oriented pins 14A and 14B are likewise mounted to a base member of like construction that is secured to lid 10 with a plurality of fasteners. A rim 18 is attached to the lower periphery of lid 10. A gasket 30, preferably santoprene, is positioned between the rim 18 and the upper periphery of tray component 20. The upper wall of lid 10 is formed with a generally rectangularly shaped recess 40. The rectangularly shaped recess 40 has at least two large apertures (more depending on the volume of the container) and one smaller aperture positioned between the larger apertures. A bimetal thermally responsive valve 42 is positioned in each of the large apertures. The bimetal valves are designed to fully open or fully close at a given temperature rather than gradually open and close over a preset temperature range. The use of bimetal valves that fully close in a step function manner, as opposed to a slow moving linear fashion as a function of temperature, significantly reduces that possibility that the container is removed from the autoclave prior to the valves reaching their completely sealed position which would result in possible contamination of the sterilized medical devices. A circular gasket 44, preferably santoprene, is positioned between each valve 42 and valve retaining housing 46. A fastener 43 attaches each bimetal valve to the valve retainer housing 46. The valve retaining housing 46 is sealed to the inner side of the upper wall of lid 10 by an oval shaped sealing gasket 48. A pressure equalizing valve 50 is positioned within the smaller aforementioned aperture and the central aperture formed in valve retaining housing 46. A filter 52 can be positioned within filter holder 54 is attached to the pressure equalizing valve 50 on the side located within the container. A top cap 60 is attached to the lid 10 within a plurality of fasteners. Spacers 62 are positioned between the bottom surface of the top cap 60 and top surface of the upper wall of lid 10.

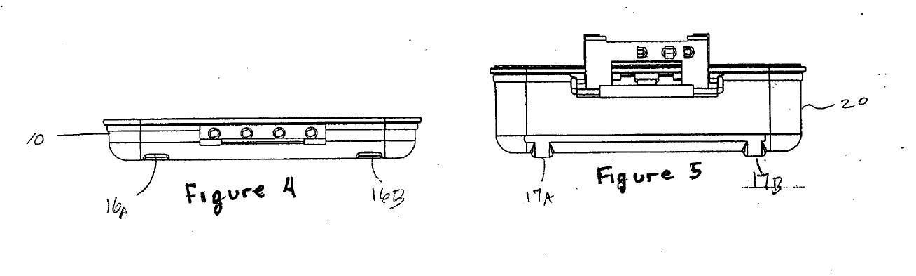

FIG. 4 is an end view of the sterilization container lid in an inverted orientation and FIG. 5 is an end view of the tray 20. Recesses 16A and 16B are shown in FIG. 4 and projections 17A and 17B are shown in FIG. 5. In total there are four projections extending from the bottom of tray 20 that are sized and configured to operatively engage the four recesses formed in the lid of an adjacent container when two or more containers are stacked. The recesses for the feet may take many different shapes from circular dots to elongated slides depicted in the drawings. All of whose function is to provide appropriate space between containers to effect stacked sterilization.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A medical instrument sterilization container comprising:
a housing having a generally rectangular bottom wall with vertical upstanding walls leading to a continuous upstanding wall peripheral edge with an open bottom;
a cooperating lid having a top wall with vertical depending walls leading to a continuous depending wall peripheral edge, said top wall having recessed area containing at least one aperture containing a bimetal thermally responsive valve and at least one aperture containing a pressure equalizing valve;
a latching mechanism for securing said cooperating lid to said housing;
wherein flash or terminal sterilization operates said bimetal thermally responsive valve to be instantly open or closed and to create a partial vacuum within the container during cooling to maintain medical instruments contained therein in a sterile environment.

2. The medical instrument sterilization container according to claim 1 including a top cap positioned above said apertures constructed and arranged to shield said valves.

3. The medical instrument sterilization container according to claim 1 including a santoprene gasket placed between said upstanding wall peripheral edge and said depending wall peripheral edge.

4. The medical instrument sterilization container according to claim 1 wherein said bimetal thermally responsive valve includes a valve retainer housing securable to said cooperating lid by a top cap, said valve retainer housing having a valve constructed from a bimetal material capable of either moving into an open position wherein pressure gas can flow through said valve or maintaining a closed position to prevent pressure gas flow in a non-linear movement.

5. The medical instrument sterilization container according to claim 4 wherein said pressure equalizing valve is mounted to said valve retainer housing.

6. The medical instrument sterilization container according to claim 1 wherein said pressure equalizing valve may include a filter on a section of said pressure equalizing valve that is located within the container.

7. The medical instrument sterilization container according to claim 1 wherein said bimetal thermally responsive valve includes a gasket positioned between said bimetal material and said valve retaining housing.

8. The medical instrument sterilization container according to claim 1 wherein said lid includes at least two recesses in the upper wall constructed and arranged to cooperate with at least two protrusions formed in the bottom wall to facilitate stacking of containers.

9. The medical instrument sterilization container according to claim 1 including at least two spacers positioned between a bottom surface of the top cap and a top surface of the upper wall of said lid.

10. A medical instrument sterilization container comprising:
a housing having a generally rectangular bottom wall with vertical upstanding walls leading to a continuous upstanding wall peripheral edge with an open bottom;
a cooperating lid having a top wall with vertical depending walls leading to a continuous depending wall peripheral edge, said top wall having recessed area containing two apertures each having a bimetal thermally responsive valve and a third aperture containing a pressure equalizing valve;
a top cap positioned above said apertures constructed and arranged to shield said valves.
a first and second latching mechanism for securing said cooperating lid to said housing;
wherein flash or terminal sterilization operates said bimetal thermally responsive valve to be either instantly open wherein pressure gas can flow through said valve or instantly closed to prevent gas flow, flash sterilization creates a partial vacuum within the container during cooling to maintain medical instruments contained therein in a sterile environment.

11. The medical instrument sterilization container according to claim 10 including a gasket placed between said upstanding wall peripheral edge and said depending wall peripheral edge.

12. The medical instrument sterilization container according to claim 10 wherein said pressure equalizing valve is mounted to said valve retainer housing.

13. The medical instrument sterilization container according to claim 10 wherein said pressure equalizing valve may include a filter on a section of said pressure equalizing valve that is located within the container.

14. The medical instrument sterilization container according to claim 10 wherein said bimetal thermally responsive valve includes a gasket positioned between said bimetal material and said valve retaining housing.

15. The medical instrument sterilization container according to claim 10 wherein said lid includes at least two recesses in the upper wall constructed and arranged to cooperate with at least two protrusions formed in the bottom wall to facilitate stacking of containers.

16. The medical instrument sterilization container according to claim 10 including at least two spacers positioned between a bottom surface of the top cap and a top surface of the upper wall of said lid.
